(19) United States
(12) Patent Application Publication
(10) Pub. No.: US 2024/0067599 A1
(43) Pub. Date: Feb. 29, 2024

(54) IODOTYROSINE DERIVATIVES AND PROCESS FOR PREPARING IODOTYROSINE DERIVATIVES

(71) Applicant: ABX ADVANCED BIOCHEMICAL COMPOUNDS - BIOMEDIZINISCHE FORSCHUNGSREAGENZIEN GMBH, Radeberg (DE)

(72) Inventors: Alexander HOEPPING, Radeberg (DE); Christoph MEYER, Radeberg (DE); Desna JOSEPH, Radeberg (DE); Stefan David KÖSTER, Radeberg (DE); Erik EISELT, Radeberg (DE)

(73) Assignee: ABX ADVANCED BIOCHEMICAL COMPOUNDS - BIOMEDIZINISCHE FORSCHUNGSREAGENZIEN GMBH, Radeberg (DE)

(21) Appl. No.: 18/255,119

(22) PCT Filed: Nov. 30, 2021
(86) PCT No.: PCT/EP2021/083649

§ 371 Date: May 31, 2023

(30) Foreign Application Priority Data
Dec. 1, 2020 (EP) 20211107.6

(51) Int. Cl. C07C 229/36; C07C 227/16

(52) U.S. Cl. CPC C07C 229/36; C07C 227/16

(57) ABSTRACT

A compound of general formula I

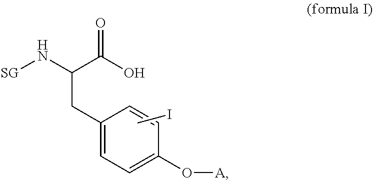

wherein A is selected from the group of an unbranched or branched alky group with 1 to 12 carbon atoms, an —R¹—O—R² group, an —R¹—Si(R³R⁴R⁵) group, an —R¹—O—Si(R³R⁴R⁵) group, a —C(O)—O—R⁹—Si(R³R⁴R⁵) group, a —CH(O—R⁶)(O—R⁷) group, an —R¹—CH(O—R⁶)(O—R⁷) group, or an —R¹—O—C(O)—O—R⁸ group; SG is a protective group; R¹ is a divalent hydrocarbon residue with 1 to 12 carbon atoms; R² is a monovalent hydrocarbon residue with 1 to 12 carbon atoms; R³, R⁴ and R⁵ each independently are a monovalent hydrocarbon residue with 1 to 12 carbon atoms; R⁶ and R⁷ each independently are a monovalent hydrocarbon residue with 1 to 12 carbon atoms; R⁸ is a monovalent hydrocarbon residue with 1 to 12 carbon atoms; and R⁹ is a divalent hydrocarbon residue with 1 to 12 carbon atoms.

### FIELD

The invention relates to iodotyrosine derivatives, in particular Fmoc-3-iodotyrosine derivatives and boc-3-iodotyrosine derivatives. Furthermore, it relates to a method for the preparation of iodotyrosine derivatives, in particular of Fmoc-3-iodotyrosine derivatives and boc-3-iodotyrosine derivatives. In addition, it relates to the use of iodotyrosine derivatives, in particular of Fmoc-3-iodotyrosine derivatives and boc3-iodotyrosine derivatives, in the synthesis of peptides.

### BACKGROUND

Modification of peptides with 3-iodotyrosine (D/L) substantially is to positively affect the properties of the iodotyrosine bearing peptides. In many cases, iodotyrosine is introduced at the N-terminal end of peptides or small molecules [4]. Due to the lipophilic nature of iodotyrosine binding properties are improved, which often results in improved receptor affinities. Examples of peptides or peptide compounds containing iodotyrosine are the theranostic peptide pair Pentixather/Pentixafor [5], HA-DOTATATE [6] or PSMA I&T [7]. Furthermore, substitution of tyrosine by iodotyrosine results in improved properties of e.g. hormones [8].

In most cases, iodotyrosine is introduced using the commercially available building blocks Fmoc-3-iodo-L-tyrosine, Fmoc-3-iodo-D-tyrosine, boc-3-iodo-D-tyrosine, or boc-3-iodo-L-tyrosine. Here, “Fmoc” designates the protective group fluorenylmethyloxycarbonyl. Boc designates the protective group tert-butyloxycarbonyl. However, using Fmoc-3-iodo-L-tyrosine, Fmoc-3-iodo-D-tyrosine, boc-3-iodo-D-tyrosine, or boc-3-iodo-L-tyrosine may be associated with undesired side reactions, since their hydroxy functionality in the para position is still sufficiently nucleophilic to be acylated by C-terminally activated amino acids. In most cases, this results in the loss of the amino acid which is no longer available for coupling. In this context, couplings with Fmoc-3-iodo-L-tyrosine, Fmoc-3-iodo-D-tyrosine, boc-3-iodo-D-tyrosine, or boc-3-iodo-L-tyrosine induced by the high reactivity of the nucleophilic hydroxy functionality of tyrosine have not proved to be practicable if there is only a low or no conversion in said reaction step.

One way to overcome said drawback is to iodize tyrosine in the peptide end product. Said processing mode typically results in two different products consisting of a mono-iodized tyrosine residue and a di-iodized tyrosine residue [9]. Both have to be separated by means of reversed-phase chromatography which makes industrial application difficult. Furthermore, said method is not suitable if more than one tyrosine entity is present in the peptide. Furthermore, said method is not suitable for industrial application, since the synthesis scale is clearly limited. In addition, the starting material has to be separated.

Cobb et al. suggest the direct iodization of completely protected Fmoc-Tyr(tBu)-OH in the presence of Ag₂SO₄ in methanol, which mainly results in Fmoc-3-iodo-Tyr(tBu)-OMe, followed by saponification. Said method is considered to be unsuitable for larger scales as needed in industrial applications. Amedio et al. [11] represented the use of boc-3-iodo-Tyr(PMB)-OH. In a further example, Kiyoyuki et al. used boc-3-iodo-Tyr(boc)-OH for the synthesis of a cyclic peptide [12]. Both protected iodotyrosine derivatives are exclusively suitable for boc chemistry. Martiny et al. synthesized Fmoc-3-iodo-Tyr(TBDMS)-OH, which has proved to be suitable for the introduction of iodized tyrosines into peptides by means of Fmoc/tBu chemistry [13]. The drawback of said specific compound is the susceptibility with respect to (weak) acids (e.g., hexafluoroisopropanol (HFIP)). Said susceptibility may result in the failure to cleave off the peptides completely protected from the resin (e.g., using HFIP). Here, “tBu” designates the protective group “tert-butyl”, “Me” methyl, “boc” the protective group tert-butoxycarbonyl, “PMB” the protective group p-methoxybenzyl, “TBDMS” the protective group “tert-butyldimethylsilyl”.

### SUMMARY

The problem of the invention is to eliminate the drawbacks according to the prior art. Provided are iodotyrosine derivatives, in particular Fmoc-3-iodotyrosine derivatives, and boc-3-iodotyrosine derivatives, which do not hinder the modification of peptides by introducing an iodotyrosine entity without the described side reactions and the cleavage of peptides modified in this way in the completely protected state of a resin.

Said problem is solved by the features of claims 1, 9, and 12. Suitable developments of the inventions result from the features of the dependent claims.

In accordance with the invention there is provided a compound of general formula I

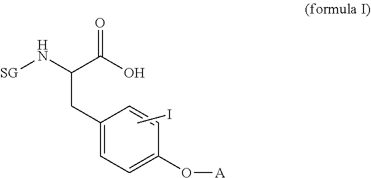

wherein
A is selected from the group consisting of an unbranched or branched alkyl group with 1 to 12 carbon atoms, an —R¹—O—R² group, an —R¹—Si(R³R⁴R⁵) group, an —R¹—O—Si(R³R⁴R⁵) group, a —C(O)—O—R⁹—Si(R³R⁴R⁵) group, a —CH(O—R⁶)(O—R⁷) group, an —R¹—CH(O—R⁶)(O—R⁷) group, an —R¹—O—C(O)—O—R⁸ group;
SG is a protective group;
R¹ is a divalent hydrocarbon residue with 1 to 12 carbon atoms;
R² is a monovalent hydrocarbon residue with 1 to 12 carbon atoms;
R³, R⁴, and R⁵ each independently are a monovalent hydrocarbon residue with 1 to 12 carbon atoms;
R⁶ and R⁷ each independently are a monovalent hydrocarbon residue with 1 to 12 carbon atoms;
R⁸ is a monovalent hydrocarbon residue with 1 to 12 carbon atoms; and
R⁹ is a divalent hydrocarbon residue with 1 to 12 carbon atoms.

Protective group SG is preferably selected from the group consisting of a fluorenylmethyloxycarbonyl group (Fmoc), a tert-butoxycarbonyl group (boc), and a benzyloxycarbonyl group. More preferably, the protective group SG is fluorenylmethyloxycarbonyl (Fmoc) or tert-butoxycarbonyl. Particularly preferred the protective group SG is fluorenylmethyloxycarbonyl (Fmoc). The protective group SG is for protecting the amino function of the tyrosine entity. The compounds of general formula I in the following are also referred to as SG iodotyrosines.

A compound of general formula I in which SG is a fluorenylmethyloxycarbonyl group (Fmoc) is a compound of general formula Ia; a compound of general formula I in which SG is a tert-butoxycarbonyl group (boc) is a compound of general formula Ib:

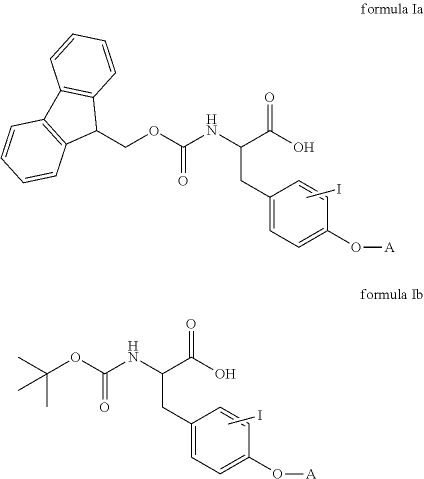

The compounds of general formula Ia in the following are also referred to as Fmoc-iodotyrosines. The compounds of general formula Ib in the following are also referred to as boc-iodotyrosines.

The compound of general formula I according to the invention comprises both each of the enantiomers in itself and mixtures of said enantiomers. Thus, the tyrosine entity of the compound of general formula I may be present in the D configuration, in the L configuration, or as a mixture of the D and L configurations. “D/L” designates a compound present in the D configuration, in the L configuration, or as a mixture of the D and L configurations.

The compound of general formula I according to the invention has an iodine atom bound to the phenyl group of the tyrosine entity.

The compounds according to the invention permit the introduction of SG-3-iodo-D-tyrosine(A)-OH or SG-3-iodo-L-tyrosine(A)-OH into peptides. The protection of the phenolic hydroxy group by the protective group A prevents the undesired side reactions which according to the prior art are associated with the unprotected hydroxy functionality. Thus, entity A prevents acylation by C-terminally activated amino acids. Thus, loss of amino acid is prevented. Moreover, now difficult couplings with SG-3-iodo-D-tyrosine(A)-OH or SG-3-iodo-L-tyrosine(A)-OH can be performed, because the phenolic hydroxy functionality is protected by means of entity A. In particular, the invention permits introduction of Fmoc-3-iodo-D-tyrosine(A)-OH or Fmoc-3-iodo-L-tyrosine(A)-OH into peptides. Furthermore, it permits introduction of boc-3-iodo-D-tyrosine(A)-OH or boc-3-iodo-L-tyrosine(A)-OH into peptides. “(A)-OH” shall indicate that the phenolic hydroxy group of the tyrosine entity is protected by protective group A, but the hydroxy group of the carboxy group is not protected. Once having introduced SG-3-iodo-D-tyrosine(A)-OH or SG-3-iodo-L-tyrosine(A)-OH into a peptide protective group A can be cleaved off.

It may be provided that the compound of general formula I is a compound of general formula I-A

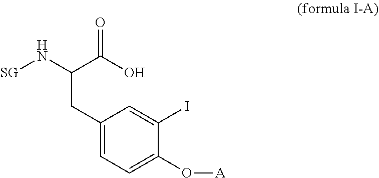

wherein A has the meanings given in claim 1. The compound of general formula I-A corresponds to the compound of general formula I except that the iodine atom is in the 3 position. A compound of general formula I-A in which SG is a fluorenylmethyloxycarbonyl group (Fmoc) is a compound of general formula Ia-A; a compound of general formula I in which SG is a tert-butoxycarbonyl group (boc) is a compound of general formula Ib-A:

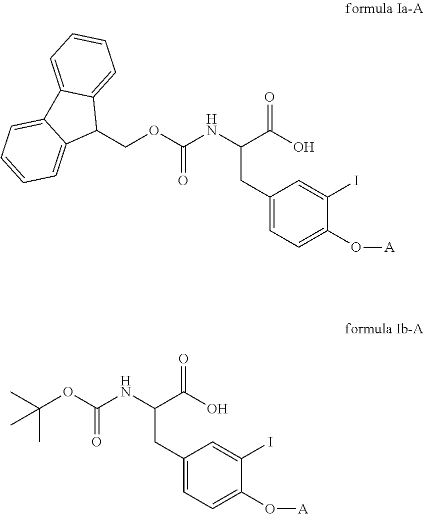

Entity A is a protective group to protect the phenolic hydroxy group of SG iodotyrosine. Entity A preferably is an ether group, a silylether group, an acetal group, or a carbonate group. In case of Fmoc-iodotyrosine entity A is preferably selected such that it is compatible with the Fmoc/tBu strategy, as applied with non-iodized Fmoc-D/L-tyrosine(tBu)-OH. In Fmoc-D/L-tyrosine(tBu)-OH the phenolic hydroxy functionality is protected by a tert-butyl group (tBu). Moreover, entity A is selected such that the compound according to the invention can be used in the production scale.

R¹ is preferably an unbranched alkylene group with 1 to 6 methylene entities. Preferably, R¹ is methylene, ethylene, or n-propylene.

R² is preferably an unbranched or branched alkyl group with 1 to 12 carbon atoms or an aryl group, with an unbranched or branched alkyl group with 1 to 12 carbon atoms being preferred.

Preferably, R³, R⁴, and R⁵ each independently are an unbranched or branched alkyl group with 1 to 2 carbon atoms or an aryl group.

Preferably, R⁶ and R⁷ each independently are an unbranched or branched alkyl group with 1 to 2 carbon atoms or an aryl group.

R⁸ is preferably an unbranched or branched alkyl group with 1 to 12 carbon atoms or an aryl group with an unbranched or branched alkyl group with 1 to 12 carbon atoms being preferred.

R⁹ is preferably an unbranched alkylene group with 1 to 6 methylene entities. Preferably, R¹ is methylene, ethylene, propylene, or butylene.

It may be provided that the entity A is selected from the group consisting of an unbranched or branched alkyl group with 1 to 12 carbon atoms, an —R¹—O—R² group, an —R¹—Si(R³R⁴R⁵) group, and an —C(O)—O—R⁹—Si(R³R⁴R⁵) group. It may be provided that the entity A is selected from the group consisting of an —R¹—O—R² group, an —R¹—Si(R³R⁴R⁵) group, an —R¹—O—Si(R³R⁴R⁵) group, a —C(O)—O—R⁹—Si(R³R⁴R⁵) group, a —CH(O—R⁶)(O—R⁷) group, an —R¹—CH(O—R⁶)(O—R⁷) group, an —R¹—O—C(O)—O—R⁸ group. It may further be provided that the entity A is selected from the group consisting of
an alkyl group with 1 to 6 carbon atoms; an —R¹—O—R² group in which R¹ is an alkylene group with 1 to 6 carbon atoms; and R² is an unbranched or branched alkyl group with 1 to 6 carbon atoms;
an —R¹—Si(R³R⁴R⁵) group in which R¹ is an alkylene group with 1 to 6 carbon atoms; and R³, R⁴, and R⁵ each independently are an unbranched or branched alkyl group with 1 to 6 carbon atoms or an aryl group; and
a —C(O)—O—R⁹—Si(R³R⁴R⁵) group in which R⁹ is an alkylene group with 1 to 6 carbon atoms; and R³, R⁴, and R⁵ each independently are an unbranched or branched alkyl group with 1 to 6 carbon atoms or an aryl group.

A compound of general formula I may be provided in which
A is selected from the group consisting of an —R¹—O—R² group, an —R¹—Si(R³R⁴R⁵) group, an —R¹—O—Si(R³R⁴R⁵) group, a —C(O)—O—R⁹—Si(R³R⁴R⁵) group, a —CH(O—R⁶)(O—R⁷) group, an —R¹—CH(O—R⁶)(O—R⁷) group, an —R¹—O—C(O)—O—R⁸ group;
SG is a protective group;
R¹ is a divalent hydrocarbon residue with 1 to 12 carbon atoms;
R² is a monovalent hydrocarbon residue with 1 to 12 carbon atoms;
R³, R⁴, and R⁵ each independently are a monovalent hydrocarbon residue with 1 to 12 carbon atoms;

R⁶ and R⁷ each independently are a monovalent hydrocarbon residue with 1 to 12 carbon atoms;
R⁸ is a monovalent hydrocarbon residue with 1 to 12 carbon atoms; and
R⁹ is a divalent hydrocarbon residue with 1 to 12 carbon atoms.

It may further be provided that the entity A is selected from the group consisting of
an —R¹—O—R² group in which R¹ is an alkylene group with 1 to 6 carbon atoms; and R² is an unbranched or branched alkyl group with 1 to 6 carbon atoms;
an —R¹—Si(R³R⁴R⁵) group in which R¹ is an alkylene group with 1 to 6 carbon atoms; and R³, R⁴, and R⁵ each independently are an unbranched or branched alkyl group with 1 to 6 carbon atoms or an aryl group; and
a —C(O)—O—R⁹—Si(R³R⁴R⁵) group in which R⁹ is an alkylene group with 1 to 6 carbon atoms; and R³, R⁴, and R⁵ each independently are an unbranched or branched alkyl group with 1 to 6 carbon atoms or an aryl group.

Preferred examples of a compound of general formula Ia-A are:
(i) 2-((((9H-fluoren-9-yl)methoxy)carbonyl)amino)-3-(3-iodo-4-(methoxymethoxy)phenyl)propionic acid, also referred to as Fmoc-D/L-Tyr(MOM)-OH, wherein Fmoc-D-Tyr(MOM)-OH is particularly preferred;
(ii) 2-((((9H-fluoren-9-yl)methoxy)carbonyl) amino)-3-(3-iodo-4-(((2-(trimethylsilyl)ethoxy)carbonyl)oxy)phenyl)propionic acid, also referred to as Fmoc-D/L-Tyr(TEOC)-OH, wherein Fmoc-D-Tyr(TEOC)-OH is particularly preferred;
(iii) 2- ((((9H-fluoren-9-yl)methoxy)carbonyl)amino)-3-(4-(2-(tert-butyldiphenylsilyl)ethoxy)-3-iodophenyl)propionic acid, also referred to as Fmoc-D/L-Tyr(TBDPSE)-OH, wherein Fmoc-D-Tyr(TBDPSE)-OH is particularly preferred; and
(iv) 2-((((9H-fluoren-9-yemethoxy)carbonyl)amino)-3-(4-(tert-butoxy)-3-iodo-phenyl)propionic acid, also referred to as Fmoc-D/L-Tyr(tBu)-OH, wherein Fmoc-D-Tyr(tBu)-OH is particularly preferred.

Preferred examples of a compound of general formula Ib-A are:
(i) 2-((tert-butoxycarbonyl)amino)-3-(3-iodo-4-(methoxymethoxy)phenyl)propionic acid, also referred to as boc-D/L-Tyr(MOM)-OH, wherein boc-D-Tyr(MOM)-OH is preferred;
(ii) 2-((tert-butoxycarbonyl) amino)-3-(3-iodo-4-(((2-(trimethylsilyl)ethoxy)carbonyl)oxy)phenyl)propionic acid, also referred to as boc-D/L-Tyr(TEOC)-OH, wherein boc-D-Tyr(TEOC)-OH is preferred;
(iii) 2-((tert-butoxycarbonyl) amino)-3-(4-(2-(tert-butyldiphenylsilyl)ethoxy)-3-iodophenyl)propionic acid, also referred to as boc-D/L-Tyr(TBDPSE)-OH, wherein boc-D-Tyr(TBDPSE)-OH is preferred; and
(iv) 2-((tert-butoxycarbonyl)amino)-3-(4-(tert-butoxy)-3-iodophenyl)propionic acid, also referred to as boc-D/L-Tyr(tBu)-OH, wherein boc-D-Tyr(tBu)-OH is preferred.

In accordance with the invention further provided is a method for the preparation of the compound of general formula I according to the invention. For that, a compound of general formula II

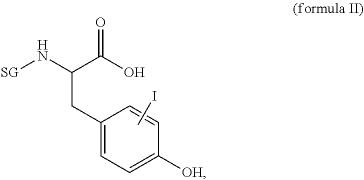

wherein SG has the meanings given in connection with general formula I, is reacted with a compound of general formula X-A, wherein X is halogen or ammonium and A has the meanings given in connection with general formula I, to a compound of general formula I

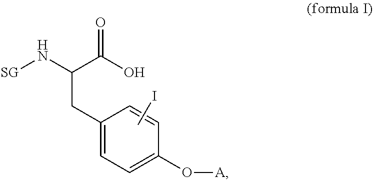

wherein SG and A have the meanings given in connection with formula I. Protective group A is introduced into the compound X-A by means of the compound of general formula II. If in the compound of general formula II SG is Fmoc, so said compound is Fmoc-iodo-D/L-tyrosine, systematically referred to as 2-((((9H-fluoren-9-yl)methoxy)carbonyl)amino)-3-(4-hydroxy-iodophenyl)propionic acid. Preferably, the compound of general formula II is Fmoc-iodo-D-tyrosine. If in the compound of general formula II SG is boc, so said compound is boc-iodo-D/L-tyrosine, systematically referred to as 2-((tert-butoxycarbonyl)amino)-3-(4-hydroxy-iodophenyl)propionic acid, wherein boc-iodo-D-tyrosine is preferred.

A particularly preferred compound of general formula II is Fmoc-3-iodo-D/L-tyrosine, systematically referred to as 2-((((9H-fluoren-9-yl)methoxy)carbonyl)amino)-3-(4-hydroxy-3-iodophenyl)propionic acid. In said compound the phenolic iodine atom is in the 3 position. Particularly preferred is Fmoc-3-iodo-D-tyrosine. A further preferred compound of general formula II is boc-3-iodo-D/L-tyrosine, systematically referred to as 2-((tert-butoxycarbonyl)amino)-3-(4-hydroxy-3-iodophenyl)propionic acid. In said compound the phenolic iodine atom is in the 3 position. Particularly preferred is boc-3-iodo-D-tyrosine.

The compound of general formula II can be prepared from a compound of general formula IV

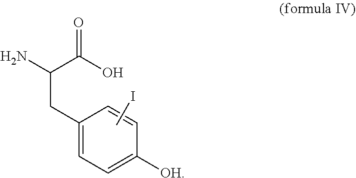

The amine function of the compound of general formula IV is protected by the introduction of a protective group SG. For that, the compound of general formula IV for example can be reacted with a 9-fluorenylmethoxycarbonyl reagent or a tert-butoxycarbonyl reagent. The 9-fluorenylmethoxycarbonyl compound can be for example (9-fluorenylmethoxycarbonyloxy)-succinimide (Fmoc-OSu). The tert-butoxycarbonyl reagent can be for example di-tert-butyldicarbonate (Boc₂O). The compound of general formula IV is D/L-iodotyrosine, wherein D-iodotyrosine is preferred.

It may be provided that the compound of general formula II is reacted with a compound of general formula X-A to obtain a compound of general formula III

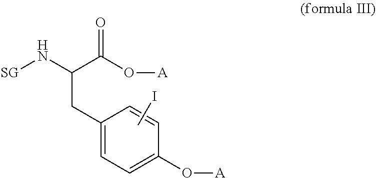

and the compound of general formula III is subsequently reacted to a compound of general formula I. Scheme 1 illustrates the inventive preparation of an inventive compound of general formula I from a compound of general formula II.

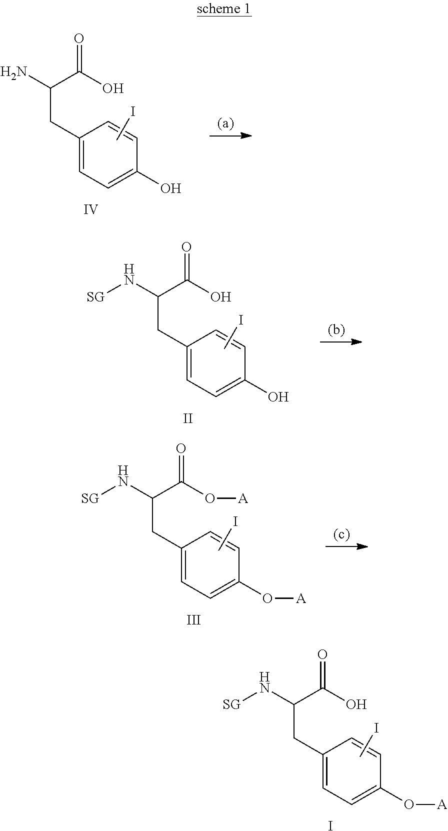

Scheme 1a illustrates the inventive preparation of an inventive compound of general formula Ia from a compound of general formula IIa. Compound IIa is a compound of general formula II in which SG is Fmoc. The method shown in scheme 1a is one embodiment of the method shown in scheme 1.

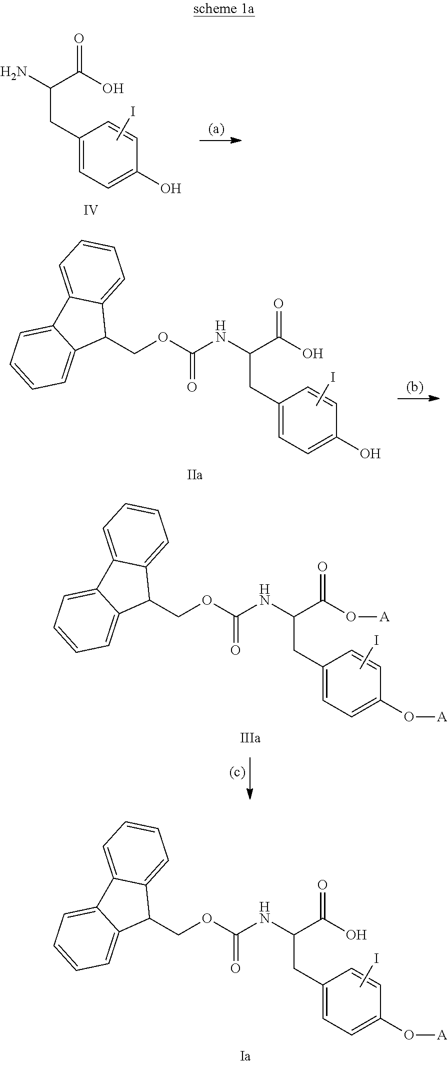

Scheme 2 illustrates the inventive preparation of an inventive compound of general formula Ia-A from 3-iodo-D/L-tyrosine. The method shown in scheme 2 is one embodiment of the method shown in scheme 1a.

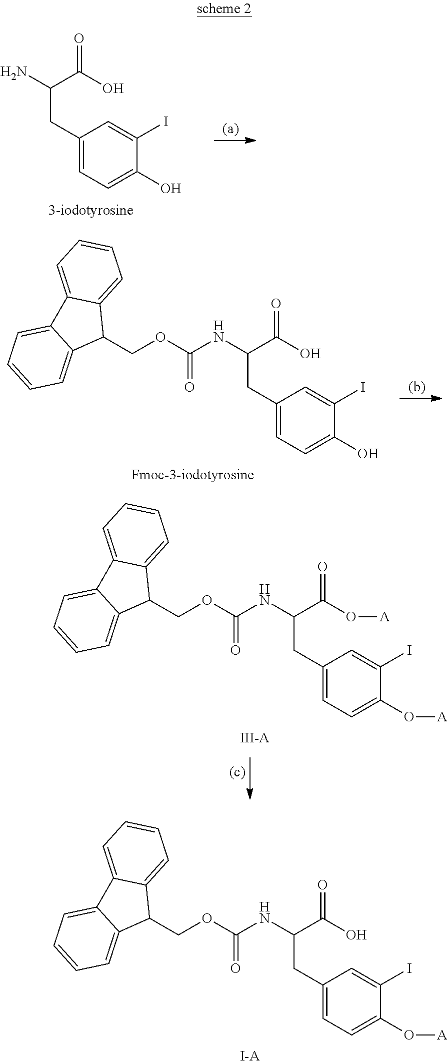

Step (a) of the method shown in scheme 1 provides the reaction of a compound of general formula IV to a compound of general formula II. Here, a protective group to protect the amine function is introduced at the N-terminus of the compound of general formula IV. For that, the compound of general formula IV can be reacted for example with (9-fluorenylmethoxycarbonyloxy)-succinimide (Fmoc-OSu) (see scheme 1a) or di-tert-butyldicarbonate (Boc₂O). The compound of general formula II corresponds to the compound of general formula IV, except that the N-terminus of the compound of general formula IV is protected by a protective group SG. Ambient temperature means a temperature in the range of 18 to 25° C.

If in step (a) of scheme 1 Fmoc is to be introduced as the protective group SG, so step (a) can be carried out at ambient pressure and ambient temperature under a protective gas, for example under argon atmosphere. In this case, to introduce Fmoc as the protective group SG step (a) is preferably carried out in a mixture of an aqueous sodium carbonate solution and 1,4-dioxane.

If in step (a) of scheme 1 boc is to be introduced as the protective group SG, so step (a) can be carried out at ambient pressure and ambient temperature in air. A protective gas is not required. In this case, step (a) is preferably carried out in a mixture of water, tetrahydrofuran, and triethylamine

Step (b) of the method shown in scheme 1 provides the reaction of a compound of general formula II to a compound of general formula III. Here, the hydroxy group at the C-terminus of the compound of general formula II and the phenolic hydroxy group are protected by an entity A. For that, the compound of general formula II is reacted with the compound X-A. The reaction can take place in an aprotic solvent such as dichloromethane (DCM) in the presence of an auxiliary base such as diisopropylethylamine (Hunig base, DIPEA) and a phase transfer catalyst such as tetrabutylammoniumchloride (TBAC1). The reaction can be carried out in a temperature range between 0° C. and ambient temperature. It can be carried out at ambient pressure and under a protective gas, for example under argon atmosphere. The compound of general formula II corresponds to the compound of general formula II, except that the hydroxy group at the C-terminus of the compound of general formula II and the phenolic hydroxy group are protected by the entity A.

Step (c) of the method shown in scheme 1 provides the reaction of a compound of general formula III to a compound of general formula I. Here, the entity A protecting the hydroxy group at the C-terminus of the compound of general formula III is cleaved off, while the entity A protecting the phenolic hydroxy group remains. Cleavage takes place in the basic range, for example in a pyridine/water mixture. The reaction can be carried out in a temperature range between 0° C. and ambient temperature. It can be carried out at ambient pressure. A protective gas is not required. The compound of general formula I corresponds to the compound of general formula III, except that there is a hydroxy group at the C-terminus of the compound of general formula I.

Further details on the method according to the invention have already been described in connection with the compound of general formula I according to the invention. Reference is made to their description.

In accordance with the invention provided is a use of a compound of general formula I according to the invention for the preparation of a peptide. The peptide prepared has at least one iodotyrosine entity. The peptide prepared can correspond to known peptides, apart from the fact that at least one, preferably exactly one tyrosine entity is replaced by a 3-iodotyrosine entity. The 3-iodotyrosine entity can be prepared by reacting a compound of general formula I with an amino acid or an amino acid sequence to obtain a peptide. Preparation of the peptide can be done by means of synthesis methods known per se, for example by means of the Merrifield synthesis. One way for peptide synthesis is described in Robert Bruce Merrifield, Solid phase peptide synthesis Journal of the American Chemical Society, Volume 85, Number 14, pp. 2149-2154. After having prepared the peptide entity A derived from the compound of general formula I is cleaved off which gives a peptide with a 3-iodotyrosine entity the phenolic OH group of which is unprotected.

Scheme 3 illustrates the preparation of a peptide of general formula V, which has an iodotyrosine entity, using a compound of general formula I.

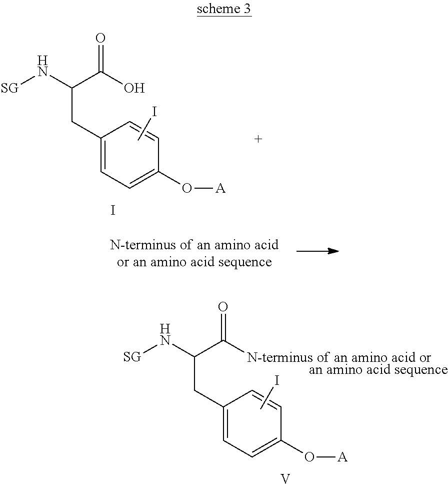

Scheme 3a illustrates the preparation of a peptide of general formula Va, which has an iodotyrosine entity, using a compound of general formula Ia. A peptide of general formula Va is a peptide of general formula V in which SG is Fmoc.

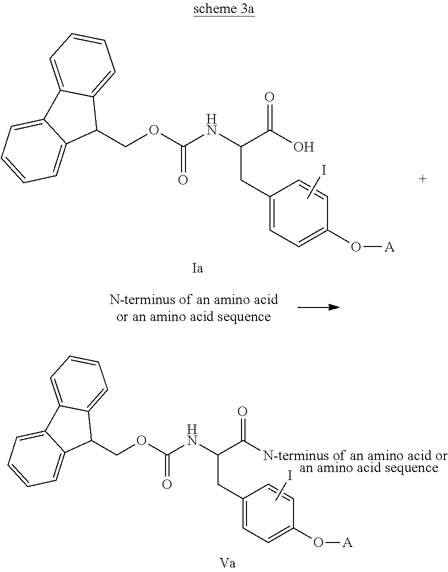

Subsequently, the protective group SG can be cleaved off, thereby converting the compound of general formula V to a compound of general formula VI, as is shown in scheme 4 and scheme 4a.

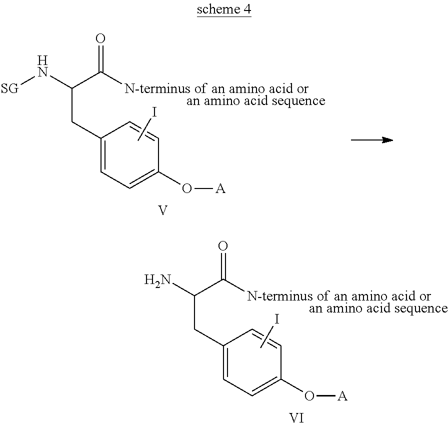

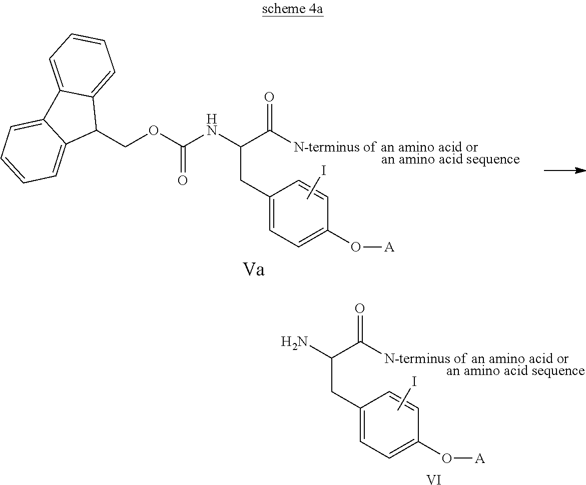

If the iodotyrosine entity shall not be a terminal entity of the peptide, so a further amino acid can be coupled to the N-terminus of the compound of general formula VI, thereby obtaining a compound of general formula VII, as is shown in scheme 5.

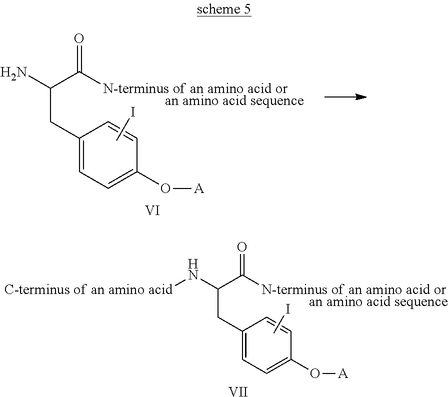

One or more additional amino acids can be bound to the further amino acid whereby a peptide of general formula VIII

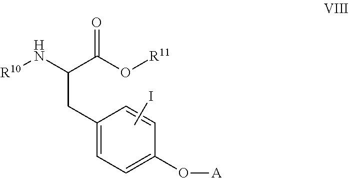

can be obtained in which A has the meanings given in connection with the compound of general formula I, R¹⁰ is hydrogen or one or more amino acid entities; and R¹¹ is hydrogen or one or more amino acid entities, with the proviso that if R¹⁰ is hydrogen, R¹¹ is not hydrogen; and that if R¹¹ is hydrogen, R¹⁰ is not hydrogen. An amino acid entity may be an entity which has an NH group or an NR¹² group at the N-terminus, wherein R¹² is a methyl group.

The compound of general formula VIII can be converted to a peptide of general formula IX by cleaving off the entity A, as is shown in scheme 6.

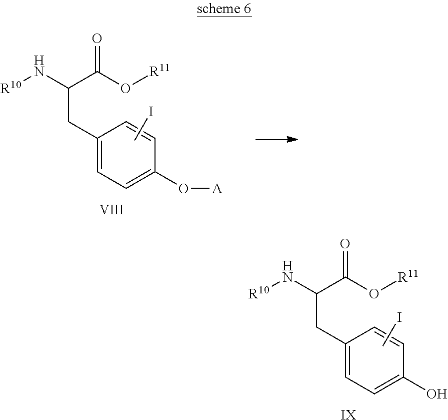

Cleavage of entity A is done in the acidic range, for example in the course of the total deprotection of the peptide. For the total deprotection of the peptide preferably an aqueous solution of trifluoroacetic acid (TFA), for example a 95% TFA solution is used. The reaction can be carried out in a temperature range between 0° C. and ambient temperature. It can be carried out at ambient pressure. A protective gas is not required. The compound of general formula IX corresponds to the compound of general formula VIII, except that entity A was cleaved off to obtain a hydroxy group.

The term “alkyl”, unless otherwise stated, in particular refers to a monovalent saturated aliphatic hydrocarbon group with a branched or unbranched carbon chain with 1 to 12 carbon atoms, preferably 1 to 8 carbon atoms and particularly preferred 1 to 6 carbon atoms. Examples of alkyl groups comprise, but are not limited to, methyl, ethyl, propyl, isopropyl, isobutyl, sec-butyl, tert-butyl, pentyl, n-hexyl, octyl, dodecyl, and the like.

The term “alkylene”, unless otherwise stated, in particular refers to a divalent saturated aliphatic hydrocarbon group with a branched or unbranched carbon chain with 1 to 12 carbon atoms, preferably 1 to 8 carbon atoms and particularly preferred 1 to 6 carbon atoms. Examples of alkylene groups comprise, but are not limited to, methylene, ethylene, propylene, butylene and the like.

The term “aryl”, unless otherwise stated, refers to a cyclic, aromatic hydrocarbon group consisting of a mono-, bi- or tricyclic aromatic ring system with 5 to 18 ring atoms, preferably 5 or 6 ring atoms. Examples of aryl groups comprise, but are not limited to, phenyl, naphthyl, anthracenyl, phenanthryl, fluorenyl, indenyl, azulenyl, biphenyl, methylenediphenyl and the like, including partially hydrogenated derivatives thereof. The aryl group, unless otherwise stated, can be mono or multivalent, for example mono or divalent.

### DETAILED DESCRIPTION

In the following, the invention is explained in more detail with the help of examples which are not intended to limit the invention.

Examples of inventive compounds are given in table 1. The compounds 1D, 2D, 3D, and 4D possess am R configuration and are derivatives of D-tyrosine. The compounds 1L, 2L, 3L, and 4L possess an S configuration and are derivatives of L-tyrosine.

TABLE 1

| | | Name |
|---|---|---|
| Compound | Structure | (abbr. chem. name) |
| 1D | 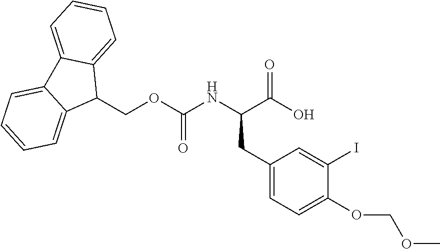 [SMILES: COCOc1ccc(C[C@@H](NC(=O)OCC2c3ccccc3-c3ccccc32)C(=O)O)cc1I] | (R)-2-((((9H-fluoren-9-yl)meth- oxy)carbonyl)amino)-3-(3-iodo- 4-(methoxymethoxy)phenyl) propionic acid (Fmoc-3-iodo-D-Tyr(MOM)- OH)) |
| 1L | 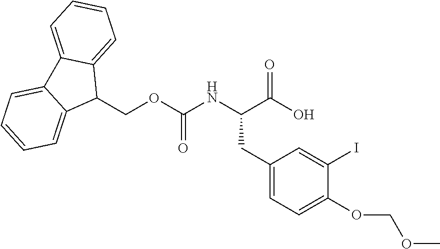 [SMILES: COCOc1ccc(C[C@H](NC(=O)OCC2c3ccccc3-c3ccccc32)C(=O)O)cc1I] | (S)-2-((((9H-fluoren-9-yl)methoxy) carbonyl)amino)-3-(3-iodo- 4-(methoxymethoxy)phenyl) propionic acid (Fmoc-3-iodo-L-Tyr(MOM)- OH)) |
| 2D | 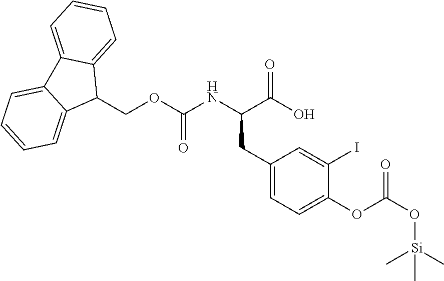 [SMILES: C[Si](C)(C)OC(=O)Oc1ccc(C[C@@H](NC(=O)OCC2c3ccccc3-c3ccccc32)C(=O)O)cc1I] | (R)-2-((((9H-Fluoren-9-yl)methoxy) carbonyl)amino)-3-(3-iodo- 4-(((2-(trimethylsilyl)ethoxy) carbonyl)oxy)phenyl)propionic acid (Fmoc-3-iodo-D-Tyr(TEOC)- OH) |
| 2L | 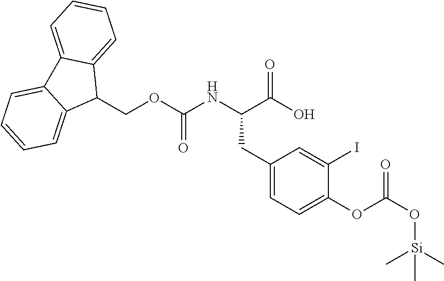 [SMILES: C[Si](C)(C)OC(=O)Oc1ccc(C[C@H](NC(=O)OCC2c3ccccc3-c3ccccc32)C(=O)O)cc1I] | (S)-2-((((9H-fluoren-9-yl)methoxy) carbonyl)amino)-3-(3-iodo- 4-(((2-(trimethylsilyl)ethoxy) carbonyl)oxy)phenyl)propionic acid (Fmoc-3-iodo-L-Tyr(TEOC)- OH) |
| 3D | 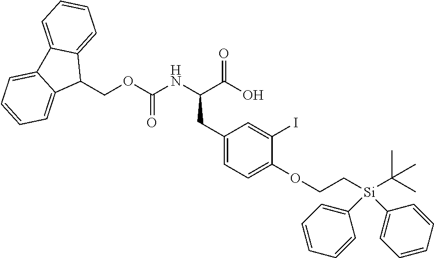 [SMILES: CC(C)(C)[Si](CCOc1ccc(C[C@@H](NC(=O)OCC2c3ccccc3-c3ccccc32)C(=O)O)cc1I)(c1ccccc1)c1ccccc1] | (R)-2-((((9H-fluoren-9-yl)methoxy) carbonyl)amino)-3-(4-(2- (tert-butyldiphenylsilyl)ethoxy)- 3-iodophenyl)propionic acid (Fmoc-3-iodo-D-Tyr(TBDPSE)- OH) |
| 3L | 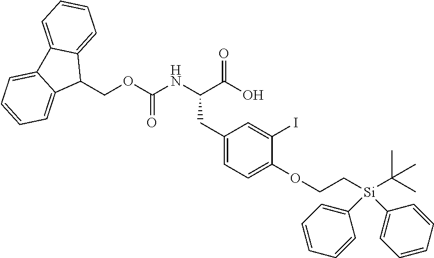 [SMILES: CC(C)(C)[Si](CCOc1ccc(C[C@H](NC(=O)OCC2c3ccccc3-c3ccccc32)C(=O)O)cc1I)(c1ccccc1)c1ccccc1] | (S)-2-((((9H-fluoren-9-yl) methoxy)carbonyl)amino)-3-(4-(2- (tert-butyldiphenylsilyl)ethoxy)- 3-iodophenyl)propionic acid (Fmoc-3-iodo-L-Tyr(TBDPSE)- OH) |
| 4D | 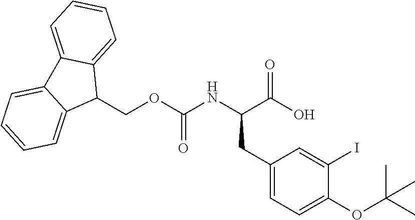 [SMILES: CC(C)(C)Oc1ccc(C[C@@H](NC(=O)OCC2c3ccccc3-c3ccccc32)C(=O)O)cc1I] | (R)-2-((((9H-fluoren-9-y1)methoxy) carbonyl)amino)-3-(4-(tert- butoxy)-3-iodophenyl)propionic acid (Fmoc-3-iodo-D-Tyr(tBu)-OH) |
| 4L | 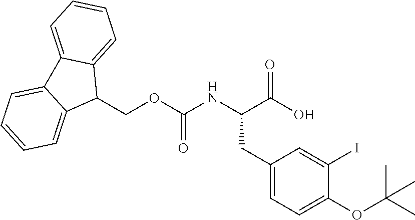 [SMILES: CC(C)(C)Oc1ccc(C[C@H](NC(=O)OCC2c3ccccc3-c3ccccc32)C(=O)O)cc1I] | (S)-2-((((9H-fluoren-9-yl)methoxy) carbonyl)amino)-3-(4-(tert- butoxy)-3-iodophenyl)propionic acid (Fmoc-3-iodo-L-Tyr(tBu)-OH) |

The compounds mentioned in table 1 are exemplary compounds of general formula I and of general formula Ia. The compounds 1D and 1L are compounds of general formula Ia in which A is an —R¹—O—R² group in which R¹ is a methylene group and R² is a methyl group. The compounds 2D and 2L are compounds of general formula Ia in which A is an —R¹—Si(R³R⁴R⁵) group in which R¹ is —CH₂—CH₂— and R³, R⁴ and R⁵ each are a methyl group. The compounds 3D and 3L are compounds of general formula Ia in which A is an —R¹—Si(R³R⁴R⁵) group in which R¹ is —CH₂—CH₂—CH₂—, R³ and R⁴ each are a phenyl group and R⁵ is a tert-butyl group. The compounds 4D and 4L are compounds of general formula Ia in which A is a tert-butyl group.

Further examples of inventive compounds are given in table 1 a. The compounds 5D, 6D, 7D and 8D possess an R configuration and are derivatives of D-tyrosine. The compounds 5L, 6L, 7L and 8L possess an S configuration and are derivatives of L-tyrosine.

TABLE 1a

| | | Name |
|---|---|---|
| Compound | Structure | (abbr. chem. name) |
| 5D | 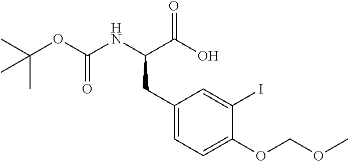 [SMILES: COCOc1ccc(C[C@@H](NC(=O)OC(C)(C)C)C(=O)O)cc1I] | (R)-2-((tert- butoxycarbonyl) amino)-3-(3-iodo- 4-(methoxymethoxy) phenyl)propionic acid (boc-3-iodo-D- Tyr(MOM)-OH)) |
| 5L | 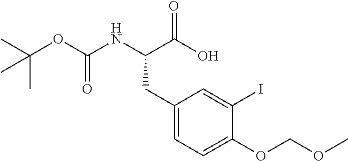 [SMILES: COCOc1ccc(C[C@H](NC(=O)OC(C)(C)C)C(=O)O)cc1I] | (S)-2-((tert-butoxycar- bonyl)amino)-3-(3-iodo- 4-(methoxymethoxy) phenyl)propionic acid (boc-3-iodo-L- Tyr(MOM)-OH)) |
| 6D | 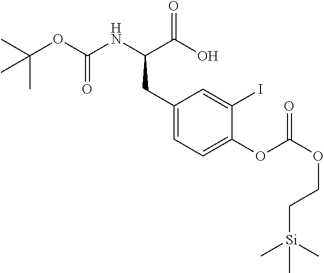 [SMILES: CC(C)(C)OC(=O)N[C@H](Cc1ccc(OC(=O)OCC[Si](C)(C)C)c(I)c1)C(=O)O] | (R)-2-((tert- butoxycarbonyl) amino)-3-(3-iodo- 4-(((2-(trimethylsilyl) ethoxy)carbonyl)oxy) phenyl)propionic acid (boc-3-iodo-D- Tyr(TEOC)-OH) |
| 6L | 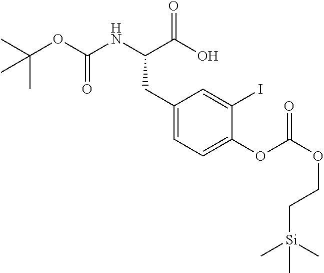 [SMILES: CC(C)(C)OC(=O)N[C@@H](Cc1ccc(OC(=O)OCC[Si](C)(C)C)c(I)c1)C(=O)O] | (S)-2-((tert- butoxycarbonyl)amino)- 3-(3-iodo- 4-(((2-(trimethylsilyl) ethoxy)carbonyl)oxy) phenyl)propionic acid (boc-3-iodo-L- Tyr(TEOC)-OH) |
| 7D | 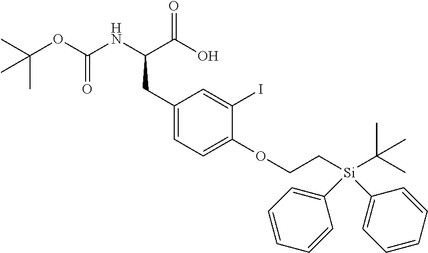 [SMILES: CC(C)(C)OC(=O)N[C@H](Cc1ccc(OCC[Si](c2ccccc2)(c2ccccc2)C(C)(C)C)c(I)c1)C(=O)O] | (R)-2- ((tert-butoxycarbonyl) amino)-3-(4-(2- (tert-butyldiphenylsilyl) ethoxy)-3-iodo- phenyl)propionic acid (boc-3-iodo-D- Tyr(TBDPSE)-OH) |
| 7L | 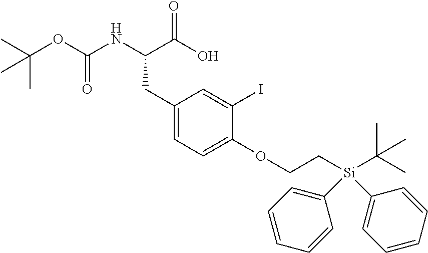 [SMILES: CC(C)(C)OC(=O)N[C@@H](Cc1ccc(OCC[Si](c2ccccc2)(c2ccccc2)C(C)(C)C)c(I)c1)C(=O)O] | (S)-2-((tert- butoxycar-bonyl)amino)- 3-(4-(2- (tert-butyldiphenylsilyl) ethoxy)-3-iodo- phenyl)propionic acid (boc-3-iodo-L- Tyr(TBDPSE)-OH) |
| 8D | 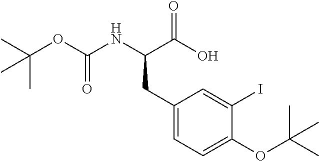 [SMILES: CC(C)(C)OC(=O)N[C@H](Cc1ccc(OC(C)(C)C)c(I)c1)C(=O)O] | (R)-2-((tert- butoxycarbonyl) amino)-3-(4-(tert- butoxy)-3-iodo- phenyl)propionic acid (boc-3-iodo-D-Tyr(tBu)- OH) |
| 8L | 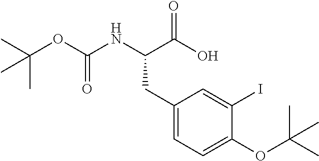 [SMILES: CC(C)(C)OC(=O)N[C@@H](Cc1ccc(OC(C)(C)C)c(I)c1)C(=O)O] | (S)-2-((tert- butoxycarbonyl) amino)-3-(4-(tert- butoxy)-3-iodo- phenyl)propionic acid (boc-3-iodo-L-Tyr(tBu)- OH) |

The compounds mentioned in table 1a are exemplary compounds of general formula I and of general formula Ib. The compounds 5D and 5L are compounds of general formula Ib in which A is an —R¹—O—R² group in which R¹ is a methylene group and R² is a methyl group. The compounds 6D and 6L are compounds of general formula Ib in which A is an —R¹—Si(R³R⁴R⁵) group in which R¹ is —CH₂—CH₂— and R³, R⁴ and R⁵ each are a methyl group. The compounds 7D and 7L are compounds of general formula Ib in which A is an —R¹—Si(R³R⁴R⁵) group in which R¹ is —CH₂—CH₂—CH₂—, R³ and R⁴ each are a phenyl group and R⁵ is a tert-butyl group. The compounds 8D and 8L are compounds of general formula Ib in which A is a tert-butyl group.

The abbreviations used in the abbreviated chemical names have the following meaning:
Boc tert-butoxy carbonyl
Fmoc 9-fluorenylmethyloxycarbonyl
MOM methoxymethyl
OH hydroxy group of the carboxyl entity
TBDPSE tert-butyldiphenylsilylethyl
TEOC 2-(trimethylsilyl)ethoxycarbonyl
D-Tyr D-tyrosine
L-Tyr L-tyrosine

### EXAMPLE 1

### Synthesis of Fmoc-3-iodo-D-Tyr(MOM)-OH (1D)

The synthesis of Fmoc-3-iodo-D-Tyr(MOM)-OH was carried out as described in scheme B1:

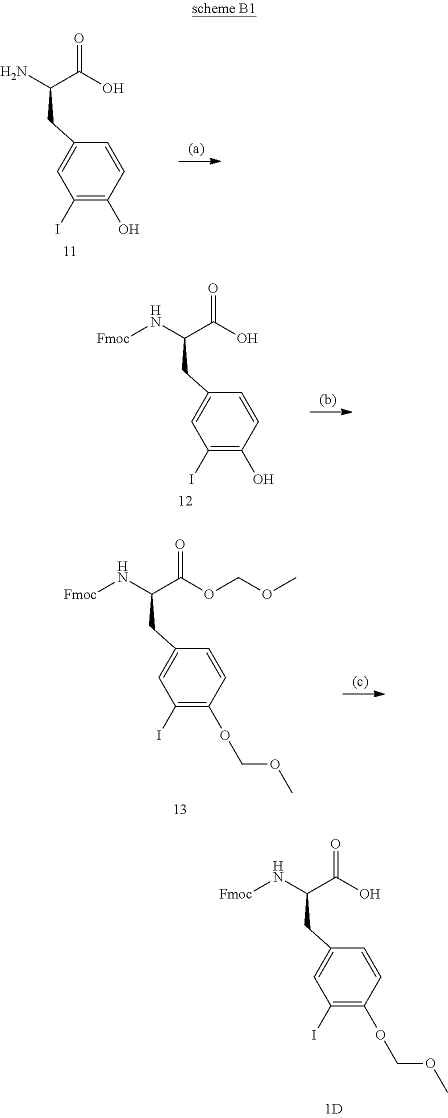

In step (a) (R)-2-amino-3-(4-hydroxy-3-iodophenyl)propionic acid 11 (also referred to as 3-iodo-D-tyrosine or 3-iodo-D-Tyr-OH) is reacted with N-(9-fluorenylmethoxycarbonyloxy)-succinimide (Fmoc-OSu) to obtain (R)-2-((((9H-fluoren-9-yl)methoxy)carbonyl)amino)-3-(4-hydroxy-3-iodophenyl)propionic acid 12 (also referred to as Fmoc-3-iodo-D-Tyr-OH). The reaction takes place in mixture of an aqueous sodium carbonate solution and 1,4-dioxane. Then, in step (b) compound 12 is reacted with methoxymethylbromide (CH₃—O—CH₂—Br) to methoxymethyl-(R)-2-((((9H-fluoren-9-yl)methoxy)carbonyl)amino)-3-(3-iodo-4-(methoxymethoxy)phenyl)propanoate 13 (also referred to as Fmoc-3-iodo-D-Tyr(MOM)-OMOM). The reaction takes place in dichloromethane (DCM) in the presence of diisopropylethylamine (DIPEA) and tetrabutylammoniumchloride (TBACl). Then, in step (c) compound 13 is reacted to the target compound 1D. The reaction is done in a mixture of tetrahydrofuran (THF), water and pyridine.

Analysis of the compound prepared was done both by HPLC analysis and LC-MS analysis.

a) Synthesis of Fmoc-3-iodo-D-Tyr-OH (12)

3-Iodo-D-Tyr-OH 11 (5 g, 16.28 mmol) was suspended in 50 ml of an aqueous Na₂CO₃ solution (1.726 g, 16.28 mmol) in an argon atmosphere. 10 ml of dioxane were added and the yellow solution was cooled in an ice water bath. Fmoc-OSu (5.492 g, 16.28 mmol) dissolved in 50 ml of 1,4-dioxane was added dropwise via a dropping funnel under an argon atmosphere. Upon addition the reaction mixture was stirred in an ice water bath for 1 hr, then at room temperature. After 17 hrs thin layer chromatography (TLC with (DCM/methanol (MeOH), 9:1 as the eluant) showed the complete conversion to the desired product Fmoc-3-iodo-D-Tyr-OH. 100 ml of H₂O were added and the mixture was cooled in an ice water bath. 30% HCl (app. 4 ml) were added until a pH of 2 to 3 was reached. The mixture was extracted with ethyl acetate (3×150 ml), the combined organic phases were washed with H₂O (2×150 ml) and saline (1×150 ml), dried over Na₂SO₄ and filtered (filter pore size 4). The solvent was removed by rotation evaporation, the residue was dried under high vacuum. Yield: 9.5 g (110%, quant.) of a white foamy solid. The crude product was used in the next step without any purification.

HPLC: t_{R}=7.26 min LC-MS: t_{R}=12.57 min, m/z=530.05 [M+H]⁺, 1059.16 [2M+H]⁺. ¹H NMR (DMSO-d⁶, 500 MHz): 12.70 (br, 1H), 10.12 (s, 1H), 7.88 (m, 2H), 7.72-7.60 (m, 4H), 7.43-7.39 (m, 2H), 7.34-7.28 (m, 2H), 7.09 (m, 1H), 6.79 (m, 1H), 4.21-4.18 (m, 3H), 4.10-5.05 (m, 1H), 2.97-2.93 (m, 1H), 2.75-2.70 (m, 1H).

b) Synthesis of Fmoc-3-iodo-D-Tyr(MOM)-OMOM (13)

Fmoc-3-iodo-D-Tyr-OH 12 (9.5 g, i.e. 8.62 g, 16.28 mmol≡100%) was suspended in 120 ml of DCM (anhydrous) under an argon atmosphere. DIPEA (5.673 ml, 32.57 mmol, 2 eq.) was added, what after stirring for 10 min at room temperature resulted in a yellow solution. TBACl (453 mg, 1.628 mmol, 0.1 eq.) was added, and the mixture was cooled in an ice-cooled water bath. Methoxymethylbromide (MOMBr) (2.658 ml, 32.57 mmol, 2 eq.) diluted in 30 ml of DCM (anhydrous) was added dropwise via a dropping funnel under an argon atmosphere (gassing). After having completed the addition the reaction mixture was stirred with ice cooling. After one hour stirring was continued for further 18 hrs at room temperature. TLC (DCM/MeOH, 50:1) showed a complete conversion. 100 ml of H₂O were added, and the mixture was vigorously stirred at room temperature. After 1 hr the phases were separated from each other in a separatory funnel. The aqueous phase was extracted several times each with 150 ml of DCM. The combined organic phases were washed with 1 N HCl (2×150 ml) and saline (150 ml), dried over Na₂SO₄ and filtered (filter pore size 4). The solvent was removed in vacuum; the remaining residue was dried in high vacuum. Yield: 11 g (109%, quant.) of a white foamy solid. The crude product was used in the next step without any purification.

HPLC: t_{R}=8.97 min LC-MS: t_{R}=14.76 min, m/z=618.12 [M+H]⁺, 1235.32 [2M+H]⁺.

c) Synthesis of Fmoc-3-iodo-D-Tyr(MOM)-OH (1D)

Fmoc-3-iodo-D-Tyr(MOM)-OMOM 13 was dissolved in 140 ml of THF (p.a.). During stirring a mixture of 400 ml of H₂O and 10 ml of pyridine was added. Ca. 100 ml of THF (p.a.) were added until a clear mixture was obtained. The mixture was refluxed under vigorous stirring in an oil bath (70° C.). After 64 hrs HPLC (214 nm) showed a complete conversion of the starting material (t_{R}=8.96 min) to the product Fmoc-3-iodo-D-Tyr(MOM)-OH. The solvent (THF) was evaporated in vacuum. The mixture was laced with 2 N HCl (app. 120 ml) under ice cooling. The pH of the solution was between pH 4 and pH 5. The mixture was extracted with DCM (3×150 ml), the combined organic phases were washed with 0.5 N HCl (2×150 ml) and saturated saline (150 ml), dried over Na₂SO₄ and filtered. The solvent was evaporated in vacuum, the remaining residue was dried in high vacuum. Yield: 9.7 g (104%, quant.) of a white foamy solid. The crude product was purified by column chromatography (yield: 4.6 g, purity by HPLC (214 nm): >95%).

m/z=574.11 [M+H]⁺, 1147.26 [2M+H]⁺. ¹H-NMR (400 MHz, CDCl₃) δ (ppm): 7.752 (d, 2H), 7.610 (s, 1H), 7.549 (m, 2H), 7.387 (t, 2H), 7.307 (m, 2H), 7.042 (d, 1H), 6.960 (d, 2H), 5.185 (s, 2H), 4.697 (m, 1H), 4.444 (m, 1H), 4.336 (m, 1H), 4.201 (m, 1H), 3.483 (s, 3H), 3.131 (m, 1H), 3.004 (m, 1H).

### EXAMPLE 2

### Synthesis of boc-3-iodo-D-Tyr(MOM)-OH (5D)

The synthesis of boc-3-iodo-D-Tyr(MOM)-OH was carried out as described in scheme B2:

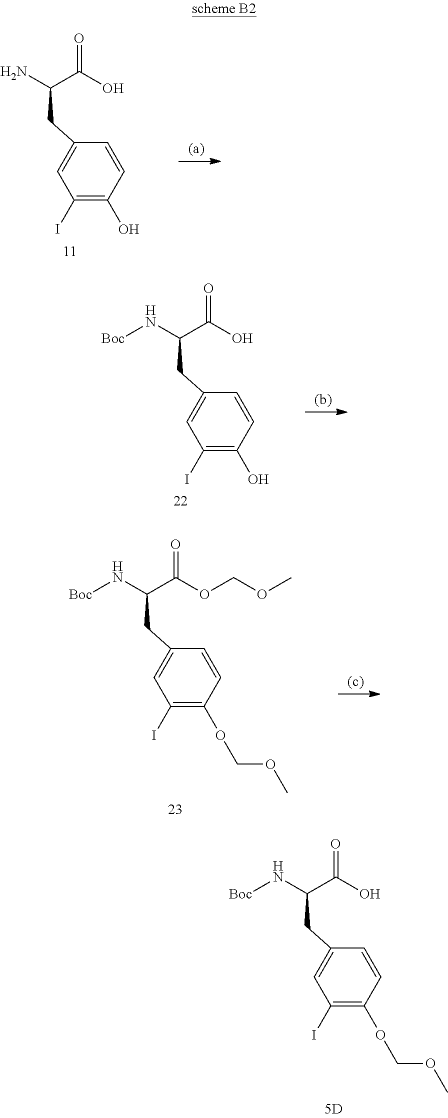 [SMILES: N[C@H](Cc1ccc(O)c(I)c1)C(=O)O ; CC(C)(C)OC(=O)N[C@H](Cc1ccc(O)c(I)c1)C(=O)O ; COCOC(=O)[C@@H](Cc1ccc(OCOC)c(I)c1)NC(=O)OC(C)(C)C ; COCOc1ccc(C[C@@H](NC(=O)OC(C)(C)C)C(=O)O)cc1I]

In step (a) (R)-2-amino-3-(4-hydroxy-3-iodophenyl)propionic acid 11 (also referred to as 3-iodo-D-tyrosine or 3-iodo-D-Tyr-OH) was reacted with di-tert-butyldicarbonate (Boc₂O) to obtain (R)-2-((tert-butoxycarbonyl)amino)-3-(4-hydroxy-3-iodophenyl)propionic acid 22 (also referred to as boc-3-iodo-D-Tyr-OH). The reaction takes place in a mixture of water, tetrahydrofuran and triethylamine Then, in step (b) compound 22 is reacted with methoxymethyl bromide (CH₃—O—CH₂—Br) to methoxymethyl-(R)-2-((tert-butoxycarbonyl)amino)-3-(3-iodo-4-(methoxymethoxy)phenyl)propanoate 23 (also referred to as boc-3-iodo-D-Tyr(MOM)-OMOM). The reaction takes place in dichloromethane (DCM) in the presence of diisopropylethylamine (DIPEA) and tetrabutylammonium chloride (TBACl). Then, in step (c) compound 23 is reacted to the target compound 5D. The reaction is done in a mixture of tetrahydrofuran (THF), water and pyridine.

a) Synthesis of boc-3-iodo-D-Tyr-OH (22)

3-Iodo-D-Tyr-OH 11 (16.28 mmol) was dissolved in 150 ml of a mixture of THF/H₂O (1:1) and TEA (4.44 ml, 32.56 mmol, 2 eq.) was added dropwise. The mixture was cooled on ice to 0° C. Boc₂O (3.63 ml, 17.9 mmol, 1.1 eq.) was melt in the water bath at 30° C. and subsequently dissolved in 20 ml of THF. The solution was transferred to a dropping funnel and added dropwise over a period of 30 minutes. After one hour the ice bath was removed and the reaction mixture was stirred overnight at room temperature. The complete conversion was controlled by mans of HPLC. THF was removed in vacuum. The aqueous solution was adjusted to pH 3-4 with 1 M HCl and extracted three times with 150 ml of ethyl acetate each. The combined organic phases were dried over sodium sulphate and the solvent was removed in vacuum. The product was dried in high vacuum. The purity of the synthesis product (boc-3-iodo-D-Tyr-OH 22) was determined by HPLC (>95%).

b) Synthesis of boc-3-iodo-D-Tyr(MOM)-OMOM (23)

Boc-3-iodo-D-Tyr-OH 22 (16.28 mmol) was dissolved in 120 ml of dry DCM. DIPEA (5.67 ml, 32.56 mmol, 2 eq.) and tetrabutylammonium chloride (0.453 g, 1.63 mmol, 0.1 eq.) were added. A solution of methoxymethyl bromide (2.657 ml, 32.56 mmol, 2 eq.) in 30 ml of DCM (anhydrous) was slowly added dropwise to an ice-cooled solution of boc-3-iodo-D-Tyr-OH over a period of 30 minutes. After one hour the ice bath was removed and subsequently stirred overnight at room temperature. After having water added and subsequently separated and dried the organic phase the organic phase was evaporated in vacuum. Complete conversion was controlled by means of HPLC. The product boc-3-iodo-D-Tyr(MOM)-OMOM 23 was identified by HPLC (>95%).

c) Synthesis of boc-3-iodo-D-Tyr(MOM)-OH (5D)

Boc-I-D-Tyr(MOM)-OMOM was dissolved in 20 ml of THF. 20 ml of a 2 M solution of LiOH in water were added and stirred for 2 hours at room temperature. THF was removed in vacuum. 300 ml of DCM and 150 ml of a 5% KHSO₄ solution were added and stirred for 5 minutes. After phase separation the aqueous phase was extracted once with 150 ml of DCM. The combined organic phases were dried over Na₂SO₄ and the solvent was removed in vacuum. The product obtained was lyophilized.

¹H-NMR (400 MHz, CDCl₃) δ (ppm): 7.619 (s, 1H), 7.103 (d, 1H), 6.993 (d, 1H), 5.216 (s, 2H), 4.968; 4.54 (m, 1H), 3.504 (s, 3H), 3.138 (m, 1H), 2.993 (m, 1H), 1.440 (s, 9H).

### EXAMPLE 3

### a) Synthesis of Tripeptides

To confirm the improved coupling properties of the compounds of general formula I-A according to the invention tripeptides have been prepared. The tripeptides prepared are shown in table 2, wherein Ac designates acetyl, Me methyl, Amb aminomethylbenzoyl and Pbf a 2,2,4,6,7-pentamethyldihydrobenzofurane-5-sulfonyl group.

For the preparation of the tripeptides P1 and P2 according to the invention either Fmoc-3-iodo-D-Tyr(MOM)-OH (compound 1D) or boc-3-iodo-D-Tyr(MOM)-OH (compound 5D) has been used. Moreover, for comparison tripeptides V1 and V2 have been prepared. Tripeptides V1 and V2 differ from tripetides P1 and P2 by the protection of the phenolic hydroxy group. In the tripetides P1 and P2 the phenolic hydroxy group is protected by a —CH₂—O—CH₃ group (MOM), while in tripeptides V1 and V2 it is not protected.

TABLE 2

| | | Designation |
|---|---|---|
| Compound | Structure | (Sequence) |
| P1 | 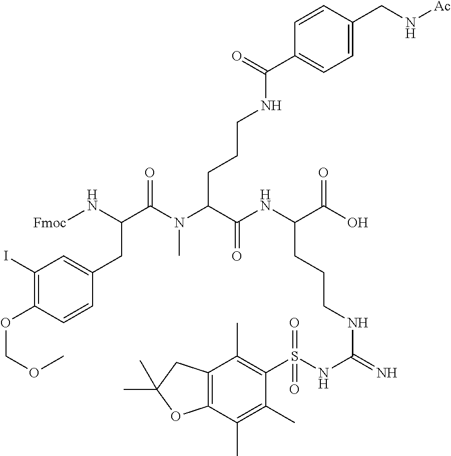 | Fmoc-3-iodo-D- Tyr(MOM)-N-Me-D- Orn(Amb-Ac)-Arg(Pbf)- OH |
| V1 | 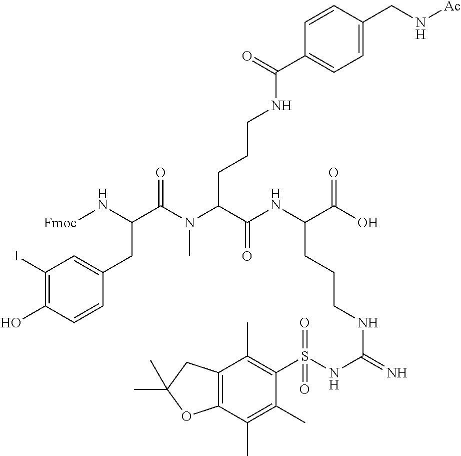 | Fmoc-3-iodo-D-Tyr-N-Me- D-Orn(Amb-Ac)-Arg(Pbf)- OH |
| P2 | 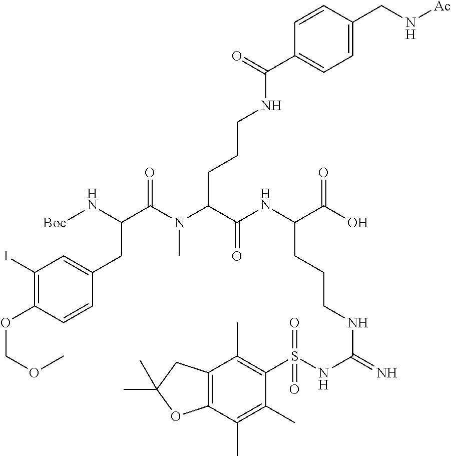 [SMILES: COCOc1ccc(CC(NC(=O)OC(C)(C)C)C(=O)N(C)C(CCCNC(=O)c2ccc(CNC(C)=O)cc2)C(=O)NC(CCCNC(=N)NS(=O)(=O)c2c(C)c(C)c3c(c2C)CC(C)(C)O3)C(=O)O)cc1I] | boc-3-iodo-D-Tyr(MOM)- N-Me-D-Orn(Amb-Ac)- Arg(Pbf)-OH |
| V2 | 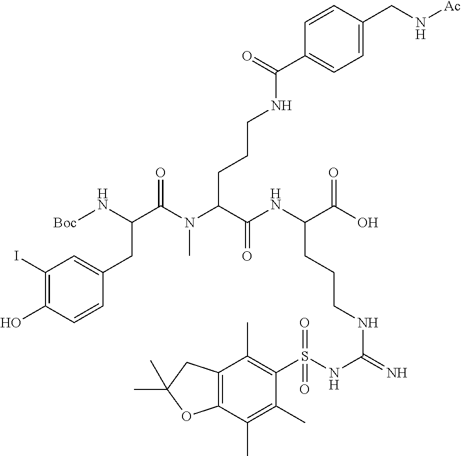 [SMILES: CC(=O)NCc1ccc(C(=O)NCCCC(C(=O)NC(CCCNC(=N)NS(=O)(=O)c2c(C)c(C)c3c(c2C)CC(C)(C)O3)C(=O)O)N(C)C(=O)C(Cc2ccc(O)c(I)c2)NC(=O)OC(C)(C)C)cc1] | boc-3-iodo-D-Tyr-N-Me- D-Orn(Amb-Ac)-Arg(Pbf)- OH |

The tripeptides have been prepared by means of the Fmoc/tBu strategy developed by Merrifield on a chlorotrityl resin which is also referred to as “Barlos resin” (Barlos, K., et al., Darstellung geschützter Peptid-Fragmente unter Einsatz substituierter Triphenylmethylharze, Tetrahedron Letters, 1989, 30(30), S. 3943-3946). This allows cleavage of completely protected peptide fragments by means of weakly acidic compounds such as hexafluoroisopropanol (HFIP). Coupling of all amino acid-like components is done by means of diisopropylcarbodiimide (DIC) and hydroxyiminocyano acetic ester (Oxyma). Cleavage of Fmoc-protective groups is done with 20% piperidine in DMF. Cleavage of the peptide from resin was done with 20% 1,1,1,3,3,3-hexafluoropropane-2-ol (HFIP) in DCM.

### b) Comparison Tests

The tripeptides P1 and P2 according to the invention and the tripeptides V1 and V2 for comparison were coupled using diisopropylcarbodiimide (DIC) and hydroxyiminocyano acetic ester (Oxyma) within 60 min.

For the preparation of tripeptide P1 Fmoc-3-iodo-D-Tyr(MOM)-OH (1D) was coupled to H—N-Me-D-Orn(Amb-Ac)-Arg(Pbf) chlorotrityl resin. Coupling was kinetically monitored. The results are shown in table 3.

TABLE 3
Synthesis of Fmoc-3-iodo-D-Tyr(MOM)-N-Me-D-Orn(Amb-Ac)-
Arg(Pbf)-OH (P1) from Fmoc-3-iodo-D-Tyr(MOM)-OH (1D) and
H-N-Me-D-Orn(Amb-Ac)-Arg(Pbf) Chlorotrityl Resin

| | | | Side |
|---|---|---|---|
| Time | Educt* | Product | Products |
| (min) | (%) | P1 (%)** | (%) *** |
| 0 | 100 | 0 | 0 |
| 15 | 73 | 26 | 0 |
| 60 | 40 | 60 | 0 |
| 120 | 14 | 86 | 0 |
| 720 | 6 | 94 | 0 |

*Educt is H-N-Me-D-Orn(Amb-Ac)-Arg(Pbf)-OH, since the reduction of Fmoc-3-iodo-D-Tyr(MOM)-OH can only be determined with some effort.
**Fmoc-3-iodo-D-Tyr(MOM)-N-Me-D-Orn(Amb-Ac)-Arg(Pbf)-OH (P1)
***It has not been determined which side products are involved.

For the preparation of tripeptide V1 Fmoc-3-iodo-D-Tyr-OH was coupled to H—N-Me-D-Orn(Amb-Ac)-Arg(Pbf) chlorotrityl resin. Coupling was kinetically monitored. The results are shown in table 4.

TABLE 4
Synthesis of Fmoc-3-iodo-D-Tyr-N-Me-D-Orn(Amb-Ac)-
Arg(Pbf)-OH (V1) from Fmoc-3-iodo-D-Tyr-OH and
H-N-Me-D-Orn(Amb-Ac)-Arg(Pbf) Chlorotrityl Resin

| | | | Side |
|---|---|---|---|
| Time | Educt* | Product | Products |
| (min) | (%) | V1 (%)** | (%)*** |
| 0 | 100 | 0 | 0 |
| 15 | 81 | 14.2 | 4.7 |
| 60 | 65.8 | 21.9 | 12.2 |
| 120 | 59.8 | 26 | 14.1 |
| 720 | 51.5 | 29 | 19.5 |

*Educt is H-N-Me-D-Orn(Amb-Ac)-Arg(Pbf)-OH, since the reduction of Fmoc-3-iodo-D-Tyr-OH can only be determined with some effort.
**Fmoc-3-iodo-D-Tyr-N-Me-D-Orn(Amb-Ac)-Arg(Pbf)-OH (V1)
***It has not been determined which side products are involved.

For the preparation of tripeptide P2 boc-3-iodo-D-Tyr(MOM)-OH (5D) was coupled to H—N-Me-D-Orn(Amb-Ac)-Arg(Pbf) chlorotrityl resin. Coupling was kinetically monitored. The results are shown in table 5.

TABLE 5
Synthesis of Boc-3-iodo-D-Tyr(MOM)-N-Me-D-Orn(Amb-Ac)-
Arg(Pbf)-OH (P2) from Boc-3-iodo-D-Tyr(MOM)-OH and
H-N-Me-D-Orn(Amb-Ac)-Arg(Pbf) Chlorotrityl Resin

| | | | Side |
|---|---|---|---|
| Time | Educt* | Product | Products |
| (min) | (%) | P2 (%)** | (%)*** |
| 0 | 100 | 0 | 0 |
| 15 | 87.3 | 12.17 | 0 |
| 60 | 37.16 | 62.84 | 0 |
| 120 | 13.37 | 86.63 | 0 |
| 720 | 0.68 | 99.32 | 0 |

*Educt is H-N-Me-D-Orn(Amb-Ac)-Arg(Pbf)-OH, since the reduction of boc-3-iodo-D-Tyr(MOM)-OH can only be determined with some effort.
**boc-3-iodo-D-Tyr(MOM)-N-Me-D-Orn(Amb-Ac)-Arg(Pbf)-OH (P2)
***It has not been determined which side products are involved.

For the preparation of tripeptide V2 boc-3-iodo-D-Tyr-OH was coupled to H—N-Me-D-Orn(Amb-Ac)-Arg(Pbf) chlorotrityl resin. Coupling was kinetically monitored. The results are shown in table 6.

TABLE 6
Synthesis of boc-3-iodo-D-Tyr-N-Me-D-Orn(Amb-Ac)-
Arg(Pbf)-OH (V2) from boc-3-iodo-D-Tyr-OH and
H-N-Me-D-Orn(Amb-Ac)-Arg(Pbf) Chlorotrityl Resin

| | | | Side |
|---|---|---|---|
| Time | Educt* | Product | Products |
| (min) | (%) | V2 (%)** | (%)*** |
| 0 | 100 | 0 | 0 |
| 15 | 93.2 | 5.7 | 1.06 |
| 60 | 73.3 | 16.1 | 10.6 |
| 120 | 65.2 | 19.8 | 15 |
| 720 | 53.74 | 18.56 | 27.7 |

*Educt is H-N-Me-D-Orn(Amb-Ac)-Arg(Pbf)-OH, since the reduction of boc-3-iodo-D-Tyr-OH can only be determined with some effort.
**boc-3-iodo-D-Tyr-N-Me-D-Orn(Amb-Ac)-Arg(Pbf)-OH
***It has not been determined which side products are involved.

Preparation of tripeptides P1 and P2 according to the invention and tripeptides V1 and V2 for comparison shows that both using Fmoc-3-iodo-D-Tyr(MOM)-OH (1D) and boc-3-iodo-D-Tyr(MOM)-OH (5D) results in the target compound of high purity and yield. Using the iodotyrosine derivatives that are unprotected in the side chain resulted in a significantly reduced yield and formation of non-specified side products. Tripeptides P1 and P2 give evidence for the increased efficiency of peptide synthesis resulting from the use of the tyrosine derivatives with protected phenolic hydroxy function according to the invention.

### EXAMPLE 4

### Synthesis of Pentixather

Pentixather could be prepared with great efficiency by means of the amino acid Fmoc-3-iodo-D-Tyr(MOM)-OH (1D), while the use of the unprotected amino acid Fmoc-3-iodo-D-Tyr-OH resulted in no or only little conversion.

### EXAMPLE 5

### Synthesis of PSMAI&T

Synthesis of the compound Glu-CO-Lys[(Sub)DLys-DPhe-DTyr(3I)-DOT-AGA]trifluoroacetate (PSMAI&T) (Wirtz, M., et al., Synthesis and in vitro and in vivo evaluation of urea-based PSMA inhibitors with increased lipophilicity. EJNMMI Research, 2018. 8(1): p. 84) run in analogy to the synthesis of pentixather with greater efficiency and significantly improved purity of the end product when using boc-3-iodo-D-Tyrosin(MOM)-OH (1D) instead of the unprotected derivative.

### CITED LITERATURE

1. Sadri, K., et al., Synthesis and biodoistribution studies of iodoine-131 D-amino acid YYK peptide as a potential therapeutic agent for labeling an anti-CD20 antibody. 2009. 52(7): p. 289-294.
2. Hallaba, E., H. El-Asrag, and Y. Abou Zeid, 131I-labelling of tyrosine by iodoine monochloride. The International Journal of Applied Radiation and Isotopes, 1970. 21(2): p. 107-110.
3. Martin, E. B., et al., Evaluation of the effect of D-amino acid incorporation into amyloid-reactive peptides. Journal of translational medicine, 2017. 15(1): p. 247-247.
4. Assoian, R. K., et al., Iodotyrosylation of peptides using tertiary-butyloxycarbonyl-l-[125I]iodootyrosine N-hydroxysuccinimide ester. Analytical Biochemistry, 1980. 103(1): p. 70-76.
5. Schottelius, M., et al., [(177)Lu]pentixather: Comprehensive Preclinical Characterization of a First CXCR4-directed Endoradiotherapeutic Agent. Theranostics, 2017. 7(9): p. 2350-2362.
6. Brogsitter, C., et al., Twins in spirit part II: DOTATATE and high-affinity DOTATATE—the clinical experience. European journal of nuclear medicine and molecular imaging, 2014. 41.
7. Weineisen, M., et al., 68Ga- and 177Lu-Labeled PSMA I&T: Optimization of a PSMA-Targeted Theranostic Concept and First Proof-of-Concept Human Studies. J Nucl Med, 2015. 56(8): p. 1169-76.
8. A, W. M., IODINATED INSULIN ANALOGUES WITH FORESHORTENED SIGNALING. 14 Dec. 2016.
9. Schottelius, M., et al., An optimized strategy for the mild and efficient solution phase iodoination of tyrosine residues in bioactive peptides. Tetrahedron Letters, 2015. 56(47): p. 6602-6605.
10. Steer, A. M., et al., A direct route for the preparation of Fmoc/OtBu protected iodootyrosine. Tetrahedron Letters, 2018. 59(27): p. 2644-2646.
11. White, J. D. and J. C. Amedio, Total synthesis of geodiamolide A, a novel cyclodepsipeptide of marine origin. The Journal of Organic Chemistry, 1989. 54(4): p. 736-738.
12. Ishiwata, H., et al., Total Synthesis of Doliculide, a Potent Cytotoxic Cyclodepsipeptide from the Japanese Sea Hare Dolabella auricularia. The Journal of Organic Chemistry, 1994. 59(17): p. 4712-4713.
13. Pedersen, M. H. F. and L. Martiny, Homogeneous deuteriodoeiodoination of iodoinated tyrosine in angiotensin-I using synthesized triethyl[2H]silane and Pd(0). 2011. 54(4): p. 191-195.

1-15. (canceled)

16. A compound of general formula I 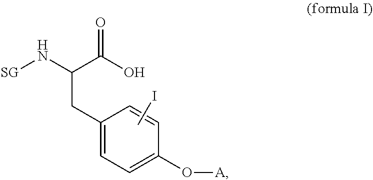
wherein
A is selected from the group consisting of an unbranched or branched alkyl group with 1 to 12 carbon atoms, an —R¹—O—R² group, an —R¹—Si(R³R⁴R⁵) group, an —R¹—O—Si(R³R⁴R⁵) group, a —C(O)—O—R⁹—Si(R³R⁴R⁵) group, a —CH(O—R⁶)(O—R⁷) group, an —R¹—CH(O—R⁶)(O—R⁷) group, an —R¹—O—C(O)—O—R⁸ group;
SG is a protective group;
R¹ is a divalent hydrocarbon residue with 1 to 12 carbon atoms;
R² is a monovalent hydrocarbon residue with 1 to 12 carbon atoms;
R³, R⁴ and R⁵ each independently are a monovalent hydrocarbon residue with 1 to 12 carbon atoms;
R⁶ and R⁷ each independently are a monovalent hydrocarbon residue with 1 to 12 carbon atoms;
R⁸ is a monovalent hydrocarbon residue with 1 to 12 carbon atoms; and
R⁹ is a divalent hydrocarbon residue with 1 to 12 carbon atoms.

17. The compound according to claim 16, wherein the compound is a compound of general formula I-A 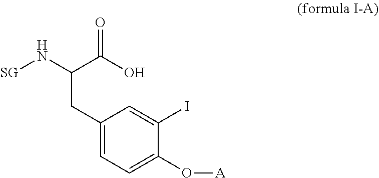
wherein A and SG have the meanings as previously defined.

18. The compound according to claim 16, wherein SG is selected from the group consisting of a fluorenylmethylenoxycarbonyl group (Fmoc), a tert-butoxycarbonyl group (boc), and a benzyloxycarbonyl group.

19. The compound according to claim 16, wherein A is selected from the group consisting of an unbranched or branched alkyl group with 1 to 12 carbon atoms, an —R¹—O—R² group, an —R¹—Si(R³R⁴R⁵) group, and a —C(O)—O—R⁹—Si(R³R⁴R⁵) group, wherein R¹, R², R³, R⁴, R⁵, and R⁹ have the meanings as previously defined.

20. The compound according to claim 16, wherein A is selected from the group consisting of an alkyl group with 1 to 6 carbon atoms; an —R¹—O—R² group in which R¹ is an alkylene group with 1 to 6 carbon atoms and R² is an unbranched or branched alkyl group with 1 to 6 carbon atoms; an —R¹—Si(R³R⁴R⁵) group in which R¹ is an alkylene group with 1 to 6 carbon atoms and R³, R⁴ and R⁵ each independently are an unbranched or branched alkyl group with 1 to 6 carbon atoms or an aryl group; and a —C(O)—O—R⁹—Si(R³R⁴R⁵) group in which R⁹ is an alkylene group with 1 to 6 carbon atoms and R³, R⁴ and R⁵ each independently are an unbranched or branched alkyl group with 1 to 6 carbon atoms or an aryl group.

21. The compound according to claim 16, wherein A is selected from the group consisting of an alkyl group with 1 to 6 carbon atoms; an —R¹—O—R² group in which R¹ is an alkylene group with 1 to 4 carbon atoms and R² is an unbranched or branched alkyl group with 1 to 6 carbon atoms; an —R¹—Si(R³R⁴R⁵) group in which R¹ is an alkylene group with 1 to 4 carbon atoms and R³, R⁴ and R⁵ each independently are an unbranched or branched alkyl group with 1 to 6 carbon atoms or an aryl group; and a —C(O)—O—R⁹—Si(R³R⁴R⁵) group in which R⁹ is an alkylene group with 1 to 6 carbon atoms and R³, R⁴ and R⁵ each independently are an unbranched or branched alkyl group with 1 to 6 carbon atoms or an aryl group.

22. The compound according to claim 16, wherein the compound is
2-((((9H-fluoren-9-yl)methoxy)carbonyl)amino)-3-(3-iodo-4-(methoxymethoxy)phenyl)propionic acid,
2-((((9H-fluoren-9-yl)methoxy)carbonyl)amino)-3-(3-iodo-4-(((2-(trimethylsilyl)ethoxy)carbonyl)oxy)phenyl)propionic acid,
2-((((9H-fluoren-9-yl)methoxy)carbonyl)amino)-3-(4-(2-(tert-butyldiphenylsilyl)ethoxy)-3-iodophenyl)propionic acid, or
2-((((9H-fluoren-9-yl)methoxy)carbonyl)amino)-3-(4-(tert-butoxy)-3-iodophenyl)propionic acid.

23. The compound according to claim 16, wherein the compound is
2-((tert-butoxycarbonyl)amino)-3-(3-iodo-4-(methoxymethoxy)phenyl)propionic acid,
2-((tert-butoxycarbonyl)amino)-3-(3-iodo-4-(((2-(trimethylsilyl)ethoxy)carbonyl)oxy)phenyl)propionic acid,
2-((tert-butoxycarbonyl)amino)-3-(4-(2-(tert-butyldiphenylsilyl)ethoxy)-3-iodophenyl)propionic acid, or
2-((tert-butoxyc arbonyl) amino)-3-(4-(tert-butoxy)-3-iodophenyl)propionic acid.

24. A method for the preparation of the compound according to claiml6, comprising reacting a compound of general formula II 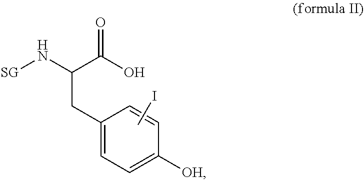
wherein SG is a protective group,
with a compound of general formula X-A, wherein
X is halogen or ammonium and
A is selected from the group consisting of an unbranched or branched alkyl group with 1 to 12 carbon atoms, an —R¹—O—R² group, an —R¹—Si(R³R⁴R⁵) group, an —R¹—O—Si(R³R⁴R⁵) group, a —C(O)—O—R⁹—Si(R³R⁴R⁵) group, a —CH(O—R⁶)(O—R⁷) group, an —R¹—CH(O—R⁶)(O—R⁷) group, an —R¹—O—C(O)—O—R⁸ group;
R¹ is a divalent hydrocarbon residue with 1 to 12 carbon atoms;
R² is a monovalent hydrocarbon residue with 1 to 12 carbon atoms;
R³, R⁴ and R⁵ each independently are a monovalent hydrocarbon residue with 1 to 12 carbon atoms;
R⁶ and R⁷ each independently are a monovalent hydrocarbon residue with 1 to 12 carbon atoms;
R⁸ is a monovalent hydrocarbon residue with 1 to 12 carbon atoms; and
R⁹ is a divalent hydrocarbon residue with 1 to 12 carbon atoms,
to form a compound of general formula I 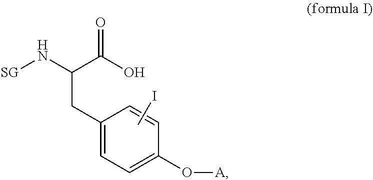
wherein A and SG have the meanings given in connection with formula II.

25. The method according to claim 24, wherein the compound of general formula II is reacted with a compound of general formula X-A to obtain a compound of general formula III 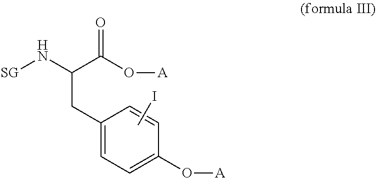
and subsequently the compound of general formula III is reacted to form a compound of general formula I.

26. The method according to claim 24, wherein the compound of general formula II is prepared from a compound of general formula IV 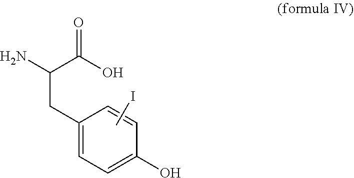
by introducing a protective group SG at the amino group of the compound of general formula IV.

27. A method for preparing a peptide, comprising reacting a compound according to claim 16.

28. The method according to claim 27, wherein the peptide is a compound of general formula IX 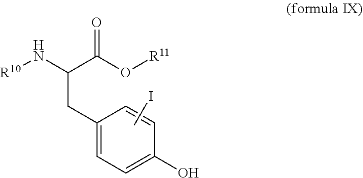
wherein
R¹⁰ is hydrogen or one or more amino acid entities; and
R¹¹ is hydrogen or one or more amino acid entities, with the proviso that, if R¹⁰ is hydrogen R¹¹ is not hydrogen and that if R¹¹ is hydrogen R¹⁰ is not hydrogen.

29. The method according to claim 27, wherein a compound of general formula VIII 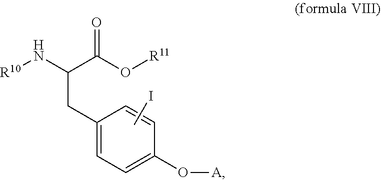
wherein
A is selected from the group consisting of an unbranched or branched alkyl group with 1 to 12 carbon atoms, an —R¹—O—R² group, an —R¹—Si(R³R⁴R⁵) group, an —R¹—O—Si(R³R⁴R⁵) group, a —C(O)—O—R⁹—Si(R³R⁴R⁵) group, a —CH(O—R⁶)(O—R⁷) group, an —R¹—CH(O—R⁶)(O—R⁷) group, an —R¹—O—C(O)—O—R⁸ group;
R¹ is a divalent hydrocarbon residue with 1 to 12 carbon atoms;
R² is a monovalent hydrocarbon residue with 1 to 12 carbon atoms;
R³, R⁴ and R⁵ each independently are a monovalent hydrocarbon residue with 1 to 12 carbon atoms;
R⁶ and R⁷ each independently are a monovalent hydrocarbon residue with 1 to 12 carbon atoms;
R⁸ is a monovalent hydrocarbon residue with 1 to 12 carbon atoms;
R⁹ is a divalent hydrocarbon residue with 1 to 12 carbon atoms; and
R¹⁰ and R¹¹ have the meanings given in connection with the compound of general formula IX;
is reacted to a compound of general formula IX.

30. The method according to claim 28, wherein the reaction is carried out in the acidic range.
